(19) United States
(12) Patent Application Publication
(10) Pub. No.: US 2024/0117302 A1
(43) Pub. Date: Apr. 11, 2024

(54) COMPOSITIONS AND METHODS FOR SMALL MOLECULE BASED EXPANSION OF PLURIPOTENT STEM CELLS

(71) Applicant: Trailhead Biosystems Inc., Cleveland, OH (US)

(72) Inventor: Michael BUKYS, Lakewood, OH (US)

(21) Appl. No.: 18/377,588

(22) Filed: Oct. 6, 2023

Related U.S. Application Data
(60) Provisional application No. 63/414,265, filed on Oct. 7, 2022.

(51) Int. Cl. C12N 5/074; C12N 5/0735

(52) U.S. Cl. CPC C12N 5/0607; C12N 5/0606; C12N 2500/25; C12N 2501/115; C12N 2501/15; C12N 2501/235; C12N 2501/415

(57) ABSTRACT

Methods and composition for growth and maintenance of Oct3/4+ SOX2+ NANOG+ pluripotent stem cells (PSCs) or generation and maintenance of CD7+ CD75+ CD77+ CD130+ F11R+ naïve PSCs in cell culture comprise the use of a small molecule-based culture media comprising an Akt pathway agonist, an FGFR pathway agonist, a JAK/STAT pathway antagonist, a PKC pathway antagonist, and an AMPK pathway agonist.

### RELATED APPLICATION

This application claims priority to U.S. Provisional Application No. 63/414,265, filed Oct. 7, 2022, the entire contents of which is hereby incorporated by reference.

### BACKGROUND OF THE INVENTION

Pluripotent stem cells have the capacity to differentiate into all cell types of the adult, making them a crucial component of regenerative medicine technologies. Therefore, the mass production of pluripotent cells with a highly consistent phenotype is of great interest. Several methods have previously been established for the growth of pluripotent stem cells in culture. Some of the most common methods include co-culture with mouse embryonic fibroblasts (MEFs) or using conditioned media that was previously exposed to MEFs. Both methods rely on the use of another cell type to secrete components into the culture media to sustain the pluripotent phenotype and are, by definition, a complex media of unknown composition.

Fully defined medias, such as the commercially available mTeSR and Essential 8 (E8) medias, are the most commonly used medias developed for the maintenance of pluripotency in culture. Both medias rely on the use of proteinaceous components using high concentrations of FGF2 in the presence of low concentrations of TGF-β to sustain a primed phenotype in human pluripotent cultures. However, protein components are prone to degradation resulting in variability over the course of their short-shelf life.

Moreover, these medias maintain human pluripotent cells in a primed phenotype, which is more representative of an epiblast population with an ectodermal bias, rather than the naïve state of pluripotent cells, which is representative of an earlier developmental state consisting of a population resembling the inner cell mass of the pre-implantation blastula. Pluripotent cells in the primed state have less differentiation potential than pluripotent cells in the naïve state.

Accordingly, while some approaches are available for maintenance and expansion of pluripotent stem cells in culture, there are limitations in these approaches and there is still a need in the art for additional methods and compositions for maintenance and expansion of pluripotent stem cells in culture, particularly that maintain the cells in a naïve state with high differentiation potential.

### SUMMARY OF THE INVENTION

The present disclosure provides compositions of fully defined media capable of sustaining a pluripotent phenotype of stem cells in culture, such as in a naïve (rather than primed) state with high differentiation potential. Thus, the compositions and methods of the present disclosure do not rely on complex media formulations of unknown components. Since this media composition is comprised of defined components, it is less subject to degradation and batch variability. In particular, this disclosure describes compositions and methods for continued maintenance of the pluripotent state in stem cells using a defined culture media composed of small molecule agonists and antagonists. This has the advantage of sustaining the pluripotent state in the absence of protein components which are subject to variability and degradation. In an embodiment, the culture media of the present invention comprises an Akt agonist, an FGF agonist, a JAK/STAT antagonist, a PKC antagonist, and an AMPK agonist.

In one aspect, a method for maintenance and expansion of Oct3/4+ SOX2+ NANOG+ pluripotent stem cells (PSCs) in cell culture comprises: culturing pluripotent stem cells (PSCs) in a culture media comprising an Akt pathway agonist, an FGFR pathway agonist, a JAK/STAT pathway antagonist, a PKC pathway antagonist, and an AMPK pathway agonist such that the culture media maintains the PSCs in a primed or naïve state comprising the markers, Oct3/4, SOX2 and NANOG.

In some embodiments, the PSCs are human PSCs (hPSCs). In other embodiments, the PSCs are induced PSCs (iPSCs). In other embodiments, the PSCs are human embryonic stem cells (hESCs). Generally, the PSCs are in a primed state (“primed PSCs”), a naïve state (“naïve PSCs”), or a combination thereof. In one embodiment, the PSCs are human CD7+ CD75+ CD77+ CD130+ F11R+ naïve pluripotent cells. In another embodiment, the PSCs express KLF2/4/5, ZFP42, ESRRB, DAPP3/5, TFCP2L1, FGF4, TBX3, CDH1, PECAM, CD31, NR5A2, and IDID1.

In an embodiment, the Akt pathway agonist is selected from the group consisting of SC79, Demethyl-Coclaurine, LM22B-10, YS-49, YS-49 monohydrate, Demethylasterriquinone B1, Recilisib, N-Oleyol glycine, NSC45586 sodium, Periplocin, CHPG sodium salt, Bilobalide, 6-hydroxyflavone, Musk ketone, SEW2871, 8-Prenylnaringenin, Razuprotafib, and combinations thereof. In a more particular embodiment, the Akt pathway agonist is SC79, which is present in the culture media at a concentration of 1 ng/ml.

In an embodiment, the FGFR agonist is FGF2 or SUN11602. In a more particular embodiment, the FGFR agonist is SUN11602, which is present in the culture media at a concentration of 5 μM.

In an embodiment, the JAK/STAT signaling antagonist is selected from the group consisting of Tofacitinib, Ruxolitinib, Baricitinib, Filgotinib, Upadacitinib, Peficitinib, Oclacitinib, Solcitinib, Decernotinib, Delgocitinib, Deucravicitinib, Abrocitinib, Lestaurtinib, Pacritinib, Fedratinib, Momelotinib, Gandotinib, Cerdulatinib, GS-829845, GSK2586184, AZD1480, R348, VX-509, GLPG0634, JSI-124, TG101348, AC-430, NS-018, CHZ868, SHR0302, INCB039110, BMS-911543, BMS-986165, PF-04965841, PF-04965842, PF-06263276, PF-06651600, and combinations thereof. In a more particular embodiment, the JAK/STAT antagonist is Tofacitinib, which is present in the culture media at a concentration of 100 nM.

In an embodiment, the PKC pathway antagonist is selected from the group consisting of Go6983, Sotrastaurin, Enzastaurin, Staurosporine, LY31615, Go 6976, GF 109203X, Ro 31-8220 mesylate, and combinations thereof. In a more particular embodiment, the PKC pathway antagonist is Go6983, which is present in the culture media at a concentration of 5 nM.

In an embodiment, the AMPK agonist is selected from the group consisting of Metformin, AICAR, Kazinol B, Marein, Amarogentin, A 769662, PF 06409577, Metformin hydrochloride, ZLN 024, ZLN 024 hydrochloride, Nilotinib, Phenformin, Nilotinib hydrochloride monohydrate, Adenosine 5′-monophosphate monohydrate, Hispidulin, MK 8722, Euphorbiasteroid, ASP4132, GSK621, EX229 (compound 991), Trans-feluric acid, O-304, MK 3903, BAM 15, ligustroflavone, ETC-1002, BC1618, IMM-H007, IM156, Chikusetsusaponin IVa, Poricoic acid A, 7-Methoxyisoflavone, Urolithin B, Danthron, Demethyleneberberine, AMPK activator 1, AMPK activator 2, AMPK activator 4, Malvidin-3-O-arabinoside chloride, RSVA 405, Etilefrin, COH-SR4, Buformin, Buformin hydrochloride, PT1, Bempedoic acid, 3a-Hydrocymogrol, Ampkinone, and combinations thereof. In one embodiment, the AMPK pathway agonist is Metformin or AICAR. In a more particular embodiment, the AMPK pathway antagonist is Metformin, which is present in the culture media at a concentration of 500 μM.

In some embodiments, the culture media is used in combination with the protein components FGF2 and/or TGF-β, such as to increase the growth rate of the pluripotent culture.

In another embodiment, the culture media further comprises a ROCK inhibitor, a TGF-β1 agonist, or both. In one embodiment, the culture media comprises a ROCK inhibitor and a TGF-β1 agonist. In one embodiment, the ROCK inhibitor is selected from the group consisting of Y27632, H1152, GSK429286A, RKI-1447, DJ4, Thiazovivin, Belumisudil, Fasudi, Hydroxyfasudil, Ripasudil, Netarsudil, and Verosudil. In a more particular embodiment, the ROCK inhibitor is Y27632, which is present in the culture media at a concentration of 10 μM.

In one embodiment, the TGF-β1 agonist is selected from the group consisting of TGF-β1, SRI-011381, Activin A, Nodal, DPS-1, and combinations thereof. In one embodiment, the TGF-β1 agonist is TGF-β1 or SRI-011381. In a more particular embodiment, the TGF-β1 agonist is TGF-β1, which is present in the culture media at a concentration of 2 ng/ml.

In one embodiment, the culture media comprises SC79, SUN11602, Tofacitinib, Go6983, and Metformin. In another embodiment, the culture media further comprises selenium, ascorbic acid, transferrin, FGF2 and TGF-β1.

In one embodiment, the culture media comprises a basal media composition selected from the group consisting of DMEM, F12, IMDM, CDM2, and combinations thereof. In another embodiment, the basal media composition is further supplemented with ascorbic acid and transferrin. In another embodiment, the basal media composition comprises F12 or IMDM media supplemented with ascorbic acid, transferrin, and penicillin-streptomycin. In a more particular embodiment, the basal media composition comprises 1:1 F12/IMDM media supplemented with 20 μg/ml ascorbic acid, 10 μg/ml transferrin and 1% penicillin-streptomycin. In another embodiment, the basal media composition comprises selenium, ascorbic acid, transferrin, FGF2 and TGF-β1.

In one embodiment, a TB5i formulation (as described herein) is supplemented into commonly used basal medias. In another embodiment, the TB5i formulation is supplemented into developed media formulations. In another embodiment, the TB5i formulation includes the addition of a ROCK inhibitor and/or a cAMP pathway activator.

In another aspect, a method for generating and maintaining a human CD7+ CD75+ CD77+ CD130+ F11R+ naïve pluripotent cell in cell culture comprising: culturing a pluripotent stem cell (PSC) in a culture media comprising an Akt pathway agonist, an FGFR pathway agonist, a JAK/STAT pathway antagonist, a PKC pathway antagonist, an AMPK pathway agonist, a ROCK inhibitor, and a TGF-β1R agonist, such that the culture media generates and maintains the human CD7+ CD75+ CD77+ CD130+ F11R+ naïve pluripotent cell in the cell culture.

In some embodiments, the PSCs are grown in an adherent culture format, such as tissue culture plates. In one embodiment, the tissue culture plates are coated with gelatin. In another embodiment, the tissue culture plates are coated with vitronectin. In yet other embodiments, the tissue culture plates are coated with MATRIGEL® or GELTREX®. In one embodiment, the TB5i formulation is used to grow pluripotent stem cells on an adherent culture format.

In other embodiments, the PSCs are grown in a suspension culture as a cell aggregate. In one embodiment, the TB5i formulation is used to grow pluripotent cells in a suspension culture as a cell aggregate.

In other embodiments, the PSCs are grown in a bioreactor. In another embodiment, the TB5i formulation is used to grow pluripotent cells in bioreactors.

### BRIEF DESCRIPTION OF THE DRAWINGS

FIG. 1 is a schematic diagram of zygote development, for determining the state of pluripotency. All the genes monitored throughout the series of HD-DoE experiments described herein are indicated within this schematic. Gene markers for the specific lineages are indicated. Optimization strategies throughout this disclosure focused on the minimization of the primed and lineage specific genes while optimizing for expression of naïve genes.

FIGS. 2A-2C show results of an HD-DoE experiment for E8 critical process parameter (CPP) media determination. FIG. 2A shows reaction conditions for each of the 96 reactions performed in the HD-DoE run. FIG. 2B shows the effectors and maximum concentrations used in the experiment. FIG. 2C depicts the basal media used in all of the experimental reactions.

FIGS. 3A-3D show results establishing CPP for basal media formulation. FIG. 3A-3C show measured genes representative of the naïve (FIG. 3A), pluripotent (FIG. 3B) and primed (FIG. 3C) states maximized within MODDE software and showing overall contribution factors for the effectors. FIG. 3D shows the average contribution factor for all effectors used, which are averaged for each representative state.

FIGS. 4A-4C show results of an HD-DoE experiment for pluripotent maintenance. FIG. 4A shows the reaction conditions for each of the 96 reactions performed in the HD-DoE run. FIG. 4B shows the effectors and max concentrations used within the experiment. FIG. 4C shows the basal media used in all of the experimental reactions.

FIGS. 5A-5B show results for NANOG optimization to reveal a small molecule pluripotent maintenance formulation. FIG. 5A shows the use of MODDE software in the optimization of key regulators of the pluripotent state as a function of NANOG gene expression. FIG. 5B shows HD-DoE informed media additives needed for the maintenance of the pluripotent state.

FIGS. 6A-6B show results demonstrating the combinatorial effects of five additives for sustaining the pluripotent state. FIG. 6A shows all of the genes marking the naïve, pluripotent, and primed states in which optimization of the effectors' relative contribution factors are presented in the heat map shown. The five components most important to maintenance of the pluripotent state are indicated. FIG. 6B shows bright-field images depicting pluripotent cultures adapting to the TB5i formulation.

FIGS. 7A-7B show comparison between TB7i and TB5i media. FIG. 7A shows a comparison of the day-to-day PSC growth using the TB7i and TB5i media formulations. Control cultures were supplemented with FGF2 and TGF-β1. FIG. 7B shows individual colonies monitored over the course of sequential days.

FIGS. 8A-8C show that inclusion of FGF2 and TGF-β within TB5i increases the naïve phenotype. FIG. 8A shows the effect of including the proteinaceous components FGF2, TGF-β and insulin in media assayed in the presence and absence of TB5i media, where T is TGF-β, F is FGF2, and I is insulin. FIG. 8B shows IHC validation of the pluripotent state. FIG. 8C shows the underlying rationale behind use of the TB5i media formulation.

FIGS. 9A-9D show that the TB5i media can sustain pluripotent cells in suspension within bioreactors. FIG. 9A shows a table showing the two media formulations used within a 100 ml PBS Vertical Wheel Bioreactor. FIG. 9B is a graph showing the overall growth of pluripotent cells within bioreactors as compared to STEMSCALE™, a commercial media for suspension cultures. FIG. 9C shows average aggregate size over the course of a 4-day reactor run. FIG. 9D shows aggregates taken from bioreactors and seeded onto a vitronectin coated plate overnight prior to immunofluorescent staining for pluripotent makers the following day.

FIG. 10 shows results for suspension culture validation of TB5i pluripotent maintenance media. The results show aggregate formation and growth within PBS bioreactors over the course of 4 days in the different media formulations shown.

### DETAILED DESCRIPTION OF THE INVENTION

Various aspects of the invention are described in further detail in the following subsections.

### I. Cells

The starting cells in the cultures are pluripotent stem cells (PSCs), including human pluripotent stem cells (hPSCs). In general, a PSC or hPSC is defined as a stem cell capable of differentiating into all cell types of the adult organism, including those characteristic of each germ cell layer (endoderm, mesoderm, and ectoderm). As used herein, a pluripotent state is used with reference to PSCs or hPSCs expressing key markers specific for e.g., induced pluripotent stem cells (iPSCs), human embryonic stem cells (hESC), such as hESC cell lines, human primed pluripotent stem cells (hpPSCs), or human naïve pluripotent stem cells (hnPSCs).

As used herein, the terms “induced pluripotent cell” and “iPSC” refer to a cell taken from a later point in development that has been induced to have expression patterns consistent with a pluripotent cell. The source of the cell can be either embryonic or adult in origin. In an embodiment, the iPSC is the iPSC cell line CR01 (NIH). Additional non-limiting examples of induced pluripotent stem cells (iPSC) include 19-11-1, 19-9-7 or 6-9-9 cells (e.g., as described in Yu, J. et al. (2009) Science 324:797-801). Non-limiting examples of human embryonic stem cell lines include ES03 cells (WiCell Research Institute) and H9 cells (Thomson, J. A. et al. (1998) Science 282:1145-1147). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2.

The terms “human embryonic stem cell and “hESC”, as used herein, refer to pluripotent cells derived from the inner cell mass of human blastocyst embryos. The term “inner cell mass” refers to a mass of cells positioned within the anterior region of the early blastula that gives rise to the entire embryo proper. Key markers for iPSCs or hESCs include, but are not limited to, OCT3/4, SOX2, NANOG and SSEA4. In addition, these cells are in a proliferative self-renewing state accompanied by expression of TERT and MKi67.

Both the naïve and the primed state are considered pluripotent cell states. As used herein, the term “primed state” refers to a pluripotent stem cell having an ectodermal bias. This is usually due to the cultures being grown in the presence of FGF2 and/or TGF-β. Most commercially available medias for human pluripotent maintenance culture cells are in this state. The term “naïve state” is used with reference to a pluripotent cell having characteristics more representative of the inner cell mass having a greater differentiation capacity and lacking the ectodermal bias characteristic of the primed state in which human pluripotent cells are normally cultured.

After their initial derivation, it was previously shown that human embryonic stem cells displayed distinct genotypic and phenotypic differences from murine pluripotent cells. Among these differences, the mouse embryonic stem cells had a faster growth rate and colonies were mounded, whereas human embryonic colonies were flat. These differences were later shown to be representative of a priming event in hESC cultures, making these cultures more representative of an epiblast population with an ectodermal bias. The culture of induced pluripotent cells followed a similar path, since the media conditions used for hESC cultures were adopted to iPSC expansion. Consequently, iPSC cultures took on a primed phenotype.

The naïve state is representative of an earlier developmental state consisting of a population resembling the inner cell mass of the pre-implantation blastula having a greater differentiation potential. The primed state is more representative of a post-implantation epiblast population with a preference to differentiate towards ectodermal lineages. The advantages of growing iPSC cells in the naïve state include a faster growth rate, increased differentiation potential and single cell clonicity. The latter could potentially remove the need for aggregate formation and the use of ROCK inhibitors during passage events. These factors underscore the benefits in developing growth media capable of expanding the naïve state in a bioreactor-based platform.

While there is a growing body of research suggesting the beneficial qualities of the naïve pluripotent state, no commercial media is known to be currently available for the expansion and maintenance of naïve pluripotent cells. In addition, most commercially available pluripotent maintenance medias rely on the incorporation of protein components that induce a primed phenotype while greatly increasing the cost of the media. For these reasons, experiments were designed to address the possibility of inducing and maintaining a naïve pluripotent state focusing on the identification of small molecules capable of mediating this transition. The use of a novel systems biology platform-informed approach capable of evaluating complex interactions in a multidimensional experimental space enables the discovery of complex combinatorial interactions between several signaling pathways for maintaining pluripotency. Through the analysis of a series of experiments, a small molecule-based culture media has been developed that is capable of sustaining PSC pluripotency generally or sustaining an increased naïve phenotype, specifically.

### II. Culture Media Components

In one aspect, the methods of the disclosure relate to maintenance and expansion of Oct3/4+ SOX2+ NANOG+ pluripotent stem cells (PSCs) in a cell culture, including PSCs in primed and/or naïve states. The methods involve the use of a small molecule-based culture media comprising specific agonists and/or antagonists of cellular signaling pathways. In some embodiments, the culture media lacks serum, lacks exogenously added growth factors, lacks animal products, is serum-free, is xeno-free and/or is feeder layer free.

As used herein, an “agonist” of a cellular signaling pathway is used with reference to an agent that stimulates (upregulates) the cellular signaling pathway. In some embodiments, stimulation of the cellular signaling pathway can be initiated extracellularly, for example by use of an agonist that activates a cell surface receptor involved in the signaling pathway (e.g., the agonist can be a receptor ligand). Additionally, or alternatively, stimulation of cellular signaling can be initiated intracellularly, for example, by use of a small molecule agonist that interacts intracellularly with one or more component(s) of the signaling pathway.

As used herein, an “antagonist” of a cellular signaling pathway is used with reference to an agent that inhibits (downregulates) the cellular signaling pathway. In some embodiments, inhibition of the cellular signaling pathway can be initiated extracellularly, for example by use of an antagonist that blocks a cell surface receptor involved in the signaling pathway. Additionally, or alternatively, inhibition of cellular signaling can be initiated intracellularly, for example, by use of a small molecule antagonist that interacts intracellularly with one or more component(s) of the signaling pathway.

Agonists and antagonists used in the methods of the disclosure are known and/or are commercially available. They are used in the culture media at concentrations effective to achieve the desired outcome, e.g., generation, expansion and/or maintenance of PSCs in primed or “naïve state, each characterized by specific corresponding markers as described herein. Non-limiting examples of suitable agonist and antagonist agents and effective concentration ranges are described further below.

In one embodiment, a method for maintenance and expansion of an Oct3/4+ SOX2+ NANOG+ pluripotent stem cell (PSC) in a cell culture comprising: culturing a pluripotent stem cell (PSC) in a culture media comprising an Akt pathway agonist, an FGFR pathway agonist, a JAK/STAT pathway antagonist, a PKC pathway antagonist, and an AMPK pathway agonist, such that the culture media maintains the PSCs in a primed or naïve state comprising the markers, Oct3/4, SOX2 and NANOG.

In some embodiments, the PSCs are human PSCs (hPSCs). In other embodiments, the PSCs are induced PSCs (iPSCs). In other embodiments, the PSCs are human embryonic stem cells (hESCs). Generally, the PSCs are in a primed state (“primed PSCs”), a naïve state (“naïve PSCs), or a combination thereof. In one embodiment, the PSCs are human CD7+ CD75+ CD77+ CD130+ F11R+ naïve pluripotent cells. In another embodiment, the PSCs express one or more of KLF2/4/5, ZFP42, ESRRB, DAPP3/5, TFCP2L1, FGF4, TBX3, CDH1, PECAM, CD31, NR5A2, and IDID1.

Agonists of the Akt pathway include agents, molecules, compounds, or substances capable of stimulating (upregulating) the signaling pathway of one or more of the serine/threonine kinase Akt family members, which include Akt1 (also designated PKB or RacPK), Akt2 (also designated PKBβ or RacPK-β) and Akt 3 (also designated PKBγ or thymoma viral proto-oncogene 3). In one embodiment, the Akt pathway agonist is a pan-Akt activator. In one embodiment, the Akt pathway agonist is selected from the group consisting of SC79, Demethyl-Coclaurine, LM22B-10, YS-49, YS-49 monohydrate, Demethylasterriquinone B1, Recilisib, N-Oleyol glycine, NSC45586 sodium, Periplocin, CHPG sodium salt, Bilobalide, 6-hydorxyflavone, Musk ketone, SEW2871, 8-Prenylnaringenin, Razuprotafib, and combinations thereof.

In one embodiment, the Akt pathway agonist is present in the culture media at a concentration within a range of 0.2-5 ng/ml, 0.3-3 ng/ml, 0.5-2.0 ng/ml, or 0.75-1.5 ng/ml. In one embodiment, the Akt pathway agonist is SC79. In one embodiment, the Akt pathway agonist is SC79, which is present in the culture media at a concentration of 0.2-5 ng/ml, 0.3-3 ng/ml, 0.5-2.0 ng/ml, or 0.75-1.5 ng/ml. In one embodiment, the Akt pathway agonist is SC79, which is present in the culture media at a concentration of 1 ng/ml.

Agonists of the FGFR pathway include agents, molecules, compounds, or substances capable of activating (upregulating) signaling through the fibroblast growth factor 2 (FGF2) signaling pathway. In one embodiment, the FGFR pathway agonist is FGF2 or SUN11602.

In one embodiment, the FGFR pathway agonist is present in the culture media at a concentration within a range of 100-500 μM, 200-400 μM, or 250-350 μM. In another embodiment, the FGFR pathway agonist is SUN11602, which is present in the culture media at a concentration in a range of 1-15 μM, 2-10 μM, or 3-7 μM. In another embodiment, the FGFR pathway antagonist is SUN11602, which is present in the culture media at a concentration of 5 μM.

Antagonists of the JAK/STAT pathway include agents, molecules, compounds, or substances capable of inhibiting (downregulating) signaling through the JAK/STAT signaling pathway. In one embodiment, the JAK/STAT pathway antagonist is selected from the group consisting of Tofacitinib, Ruxolitinib, Baricitinib, Filgotinib, Upadacitinib, Peficitinib, Oclacitinib, Solcitinib, Decernotinib, Delgocitinib, Deucravicitinib, Abrocitinib, Lestaurtinib, Pacritinib, Fedratinib, Momelotinib, Gandotinib, Cerdulatinib, GS-829845, GSK2586184, AZD1480, R348, VX-509, GLPG0634, JSI-124, TG101348, AC-430, NS-018, CHZ868, SHR0302, INCB039110, BMS-911543, BMS-986165, PF-04965841, PF-04965842, PF-06263276, PF-06651600, and combinations thereof.

In one embodiment, the JAK/STAT pathway antagonist is present in the culture media at a concentration within a range of 25-250 nM, 50-150 nM, or 75-125 nM. In another embodiment, the JAK/STAT pathway antagonist is Tofacitinib, which is present in the culture media at a concentration of 25-250 nM, 50-150 nM, or 75-125 nM. In another embodiment, the JAK/STAT pathway antagonist is Tofacitinib, which is present in the culture media at a concentration of 100 nM.

Antagonists of the PKC pathway include agents, molecules, compounds, or substances capable of inhibiting (downregulating) signaling through the PKC signaling pathway. In one embodiment, the PKC pathway antagonist is selected from the group consisting of Go6983, Sotrastaurin, Enzastaurin, Staurosporine, LY31615, Go 6976, GF 109203X, Ro 31-8220 mesylate, and combinations thereof.

In one embodiment, the PKC pathway antagonist is present in the culture media at a concentration within a range of 2-10 nM, 2.5-7.5 nM, 3-6.50 nM, or 4-6 nM. In another embodiment, the PKC pathway antagonist is Go6983, which is present in the culture media at a concentration of 2-10 nM, 2.5-7.5 nM, 3-6.50 nM, or 4-6 nM. In another embodiment, the PKC pathway antagonist is Go6983, which is present in the culture media a concentration of 5 nM.

Agonists of the AMPK pathway include agents, molecules, compounds, or substances capable of activating (upregulating) signaling through the AMPK signaling pathway. In one embodiment, the AMPK pathway agonist is selected from the group consisting of Metformin, AICAR, Kazinol B, Marein, Amarogentin, A 769662, PF 06409577, Metformin hydrochloride, ZLN 024, ZLN 024 hydrochloride, Nilotinib, Phenformin, Nilotinib hydrochloride monohydrate, Adenosine 5′-monophosphate monohydrate, Hispidulin, MK 8722, Euphorbiasteroid, ASP4132, GSK621, EX229 (compound 991), Trans-feluric acid, O-304, MK 3903, BAM 15, ligustroflavone, ETC-1002, BC1618, IM-H007, IM156, Chikusetsusaponin IVa, Poricoic acid A, 7-Methoxyisoflavone, Urolithin B, Danthron, Demethyleneberberine, AMPK activator 1, AMPK activator 2, AMPK activator 4, Malvidin-3-O-arabinoside chloride, RSVA 405, Etilefrin, COH-SR4, Buformin, Buformin hydrochloride, PT1, Bempedoic acid, 3a-Hydrocymogrol, Ampkinone, and combinations thereof.

In one embodiment, the AMPK pathway antagonist is present in the culture media at a concentration within a range of 200-1000 μM, 250-750 μM, 300-650 μM, or 400-600 μM. In another embodiment, the AMPK pathway antagonist is Metformin, which is present in the culture media at a concentration of 200-1000 μM, 250-750 μM, 300-650 μM, or 400-600 μM. In another embodiment, the AMPK pathway antagonist is Metformin, which is present in the culture media at a concentration of 500 μM.

In one embodiment, the small molecule culture media comprises a basal media composition supplemented with SC79, SUN11602, Tofacitinib, Go6983, and Metformin. In a more particular embodiment, the culture media includes a basal media composition supplemented with 1 ng/ml SC79, 5 μM SUN11602, 100 nM Tofacitinib, 5 nM Go6983, and 500 μM Metformin.

In one embodiment, the small molecule culture media comprises a basal media composition selected from the group consisting of DMEM, F12, IMDM, CDM2, and combinations thereof. In another embodiment, the small molecule culture media comprises a basal media composition supplemented with ascorbic acid and transferrin.

In one embodiment, the small molecule culture media comprises a basal media composition comprising F12 or IMDM media supplemented with ascorbic acid, transferrin, and penicillin-streptomycin. In a more particular embodiment, the basal media composition in the culture media comprises a 1:1 F12/IMDM media supplemented with 20 μg/ml ascorbic acid, 10 μg/ml transferrin and 1% penicillin-streptomycin. In another embodiment, the small molecule culture media comprises a basal media composition comprising a 1:1 F12/IMDM media supplemented with 20 μg/ml ascorbic acid, 10 μg/ml transferrin and 1% penicillin-streptomycin, where the basal media composition is further supplemented with either 100 ng/ml FGF2 and 2 ng/ml TGF-β1 (hereinafter “the TB5i media formulation”) or 10 μM Y27632 and 1 μM forskolin (hereinafter “the TB7i media formulation”).

In some embodiments, the small molecule-based culture media comprises a basal media composition comprising selenium, ascorbic acid, transferrin, FGF2, and TGF-β1.

In some embodiments, the small molecule-based culture media is used in combination with the protein components FGF2 and/or TGF-β1 to increase the growth rate of the pluripotent stem cell culture.

In some embodiments, the culture media further comprises a Rho kinase inhibitor (i.e., ROCK inhibitor), a TGF-β1 pathway agonist, or both. In one embodiment, the culture media comprises a ROCK inhibitor and a TGF-β1 pathway agonist.

ROCK inhibitors include agents, molecules, compounds, or substances capable of inhibiting (downregulating) signaling through the Rho kinase pathway. In one embodiment, the ROCK inhibitor is selected from the group consisting of Y27632, H1152, GSK429286A, RKI-1447, DJ4, Thiazovivin, Belumisudil, Fasudi, Hydroxyfasudil, Ripasudil, Netarsudil, and Verosudil.

In one embodiment, the ROCK inhibitor is present in the culture media at a concentration within a range of 2-50 μM, 3-30 μM, 5-20 μM, or 7.5-15 μM. In another embodiment, the ROCK inhibitor is Y27632, which is present in the culture media at a concentration of 2-50 μM, 3-30 μM, 5-20 μM, or 7.5-15 μM. In another embodiment, the ROCK inhibitor is Y27632, which is present in the culture media at a concentration of 10 μM.

TGF-β1 pathway agonists include agents, molecules, compounds, or substances capable of activating (upregulating) signaling through the TGF-β1 signaling pathway. In some embodiments, the TGF-β1 agonist is selected from the group consisting of TGF-β1, SRI-011381, Activin A, Nodal, DPS-1, and combinations thereof. In one embodiment, the TGF-β1 agonist is TGF-β1 or SRI-011381.

In one embodiment, the TGF-β1 pathway agonist is present in the culture media at a concentration within a range of within a range of 0.4-10 ng/ml, 0.6-6 ng/ml, 1-4 ng/ml, or 1.5-3 ng/ml. In one embodiment, the TGF-β1 pathway agonist is TGF-β1. In one embodiment, the TGF-β1 pathway agonist is TGF-β1, which is present in the culture media at a concentration of within a range of 0.4-10 ng/ml, 0.6-6 ng/ml, 1-4 ng/ml, or 1.5-3 ng/ml. In one embodiment, the TGF-β1 pathway agonist is TGF-β1, which is present in the culture media at a concentration of 2 ng/ml.

In another aspect, a method for generating and maintaining a human CD7+ CD75+ CD77+ CD130+ F11R+ naïve pluripotent cell in cell culture comprising: culturing a pluripotent stem cell (PSC) in a culture media according to the present application comprising an Akt pathway agonist, an FGFR pathway agonist, a JAK/STAT pathway antagonist, a PKC pathway antagonist, an AMPK pathway agonist, a ROCK inhibitor, and a TGF-β1R agonist, such that the culture media generates and maintains the human CD7+ CD75+ CD77+ CD130+ F11R+ naïve pluripotent cell in the cell culture.

In another aspect, the present application provides a small molecule-based culture media for growth, maintenance, and expansion of Oct3/4+ SOX2+ NANOG+ pluripotent stem cells (PSCs), as well as for generation, growth, maintenance, and expansion of human CD7+ CD75+ CD77+ CD130+ F11R+ naïve pluripotent cells in cell culture, as described herein.

In one embodiment, the small molecule-based culture media comprises an Akt pathway agonist, an FGFR pathway agonist, a JAK/STAT pathway antagonist, a PKC pathway antagonist, and an AMPK pathway agonist. As described above, a basal media composition of the present application is supplemented with the foregoing agonists and antagonists in the above-described concentrations.

In one embodiment, the Akt pathway agonist is SC79, which is present in the culture media at a concentration of 1 ng/ml. In another embodiment, the FGFR agonist is SUN11602, which is present in the culture at a concentration of 5 μM. In another embodiment, the JAK/STAT antagonist is Tofacitinib, which is present in the culture media at a concentration of 100 nM. In another embodiment, the PKC pathway antagonist is Go6983, which is present in the culture media at a concentration of 5 nM. In another embodiment, the AMPK pathway agonist is Metformin, which is present in the culture media at a concentration of 500 μM. In a preferred embodiment, the small molecule culture media comprises a basal media composition is supplemented with 1 ng/ml SC79, 5 μM SUN11602, 100 nM Tofacitinib, 5 nM Go6983, and 500 μM Metformin.

In another embodiment, the small molecule culture media comprises a basal media composition, which is further supplemented with a ROCK inhibitor, where the ROCK inhibitor is Y27632, and where Y27632 is present in the culture media at a concentration of 10 μM.

In another embodiment, the small molecule culture media comprises a basal media composition, which is further supplemented with a TGF-β1 agonist, where the TGF-β1 agonist is TGF-β1, and where TGF-β1 is present in the culture media at a concentration of 2 ng/ml.

In a preferred embodiment, the small molecule culture media comprises a basal media composition comprising 1:1 F12/IMDM media supplemented with 20 μg/ml ascorbic acid, 10 μg/ml transferrin and 1% penicillin-streptomycin, which is supplemented with 1 ng/ml SC79, 5 μM SUN11602, 100 nM Tofacitinib, 5 nM Go6983, and 500 μM Metformin, and is further supplemented with either 100 ng/ml FGF2 and 2 ng/ml TGF-β1 (i.e., TB5i media formulation) or with 10 μM Y27632 and 1 μM forskolin (i.e., TB7i media formulation).

When an agonist or antagonist is used in more than one step of the method, in one embodiment, the same agonist or antagonist is used for each step in which the agent is present in the culture media. In another embodiment, a different agonist or antagonist affecting the same signaling pathway is used in different steps of the method.

When an agonist or antagonist is used in more than one step of the method, in one embodiment, the same concentration of the agonist or antagonist is used for each step in which the agent is present in the culture media. In another embodiment, different concentrations of the same agonist or antagonist are used in different steps of the method.

### III. Culture Conditions

In combination with the chemically defined and optimized culture media described in subsection II above, the methods for maintenance, expansion, and generation of PSC cells described above utilize standard culture conditions established in the art for cell culture. For example, cells can be cultured at 37° C. and 5% CO₂ conditions.

In some embodiments, the PSCs are cultured with daily media changes using the culture media described herein on adherent culture formats using standard culture vessels or plates, such as 6-well, 24-well, or 96-well tissue culture (TC) plates. In certain embodiments, the PSCs are coated with an extracellular matrix material. In one embodiment, the TC plates are coated with gelatin. In another embodiment, the TC plates are coated with vitronectin. In another embodiment, the TC plates are coated with MATRIGEL®. In another embodiment, the TC plates are coated with GELTREX®.

The culture media (e.g., TB5i formulation) described herein has been shown to be effective for growing and maintaining adherent cultures grown in tissue culture plates. In an exemplary embodiment, a PSC culture, such as the CR01 iPSC line, is grown and maintained on vitronectin coated 6-well TC plates using a culture media of the present application, such as TB5i media. PSC cultures are generally passaged every 3-4 days and treated with agents for disrupting cell-to-cell adhesion, such as EDTA, or digestion enzymes, such as collagenase, accutase, trypsin, or TyrPLE. This can be accomplished by removing the media and washing each well of the TC plate with 2 ml of PBS. A 3-minute incubation in the presence of 5 mM EDTA can then be performed at 37 degrees C. Wells are then aspirated, and the cells are washed off from the plates and seeded in fresh media. Each well passaged is generally seeded onto 6 wells of a newly vitronectin-coated TC plate resulting in a 1 to 6 expansion of the iPSC line.

In some embodiments, suspension cultures of PSCs can be grown as cell aggregates in bioreactors as further described in the Example 3 below. In an exemplary embodiment, the TB5i media formulation can be used to grow PSCs in suspension cultures in 100 ml PBS VW bioreactors at 60 RPM for 5 sequential days with a demi-depletion on day 1 and every 2nd day after that.

### IV. Uses

The culture media described herein, such as TB5i media formulation, can be used for the maintenance and expansion of Oct3/4+ SOX2+ NANOG+ PSCs in cell culture, including those in both a primed or naïve state of differentiation. In addition, the culture media can be used to generate and maintain human CD7+ CD75+ CD77+ CD130+ F11R+ naïve pluripotent cells from a primed PSC state in cell culture. The ability to maintain and expand pluripotent cells in culture using the compositions and methods of the disclosure allows for obtention of large quantities of these cells, including for a wide variety of regenerative medicine purposes.

### V. Compositions

In other aspects, the disclosure provides compositions related to methods for maintenance and expansion of an Oct3/4+ SOX2+ NANOG+ pluripotent stem cell (PSC) in cell culture, including culture media and cell cultures, as well as compositions related to methods for generating and maintaining a human CD7+ CD75+ CD77+ CD130+ F11R+ naïve pluripotent cell in cell culture, including culture media and cell cultures.

Accordingly, in one aspect, the disclosure provides a culture media for maintenance and expansion of an Oct3/4+ SOX2+ NANOG+ pluripotent stem cell (PSC) comprising an Akt pathway agonist, an FGFR pathway agonist, a JAK/STAT pathway antagonist, a PKC pathway antagonist and an AMPK pathway agonist.

In certain embodiments, the culture media further comprises a ROCK inhibitor, a TGF-β1 agonist, or both.

In certain embodiments, the culture media further comprises a basal media composition. In certain embodiments, the basal media composition comprises a media selected from the group consisting of DMEM, F12, IMDM, CDM2, and combinations thereof. In certain embodiments, the basal media composition is further supplemented with ascorbic acid and transferrin. In certain embodiments, the basal media composition comprises F12 or IMDM media supplemented with ascorbic acid, transferrin, and penicillin-streptomycin. In certain embodiments, the basal media composition comprises selenium, ascorbic acid, transferrin, FGF2, and TGF-β1.

In another aspect, the disclosure provides an isolated cell culture comprising Oct3/4+ SOX2+ NANOG+ PSCs cultured in one of the media formulations disclosed herein. Accordingly, in an embodiment, the disclosure provides an isolated cell culture comprising Oct3/4+ SOX2+ NANOG+ PSCs cultured in a culture media comprising an Akt pathway agonist, an FGFR pathway agonist, a JAK/STAT pathway antagonist, a PKC pathway antagonist and an AMPK pathway agonist. In certain embodiments, the culture media further comprises a ROCK inhibitor, a TGF-β1 agonist, or both. In certain embodiments, the culture media further comprises a basal media composition. In certain embodiments, the basal media composition comprises a media selected from the group consisting of DMEM, F12, IMDM, CDM2, and combinations thereof. In certain embodiments, the basal media composition is further supplemented with ascorbic acid and transferrin. In certain embodiments, the basal media composition comprises F12 or IMDM media supplemented with ascorbic acid, transferrin, and penicillin-streptomycin. In certain embodiments, the basal media composition comprises selenium, ascorbic acid, transferrin, FGF2, and TGF-β1.

The present invention is further illustrated by the following examples, which should not be construed as further limiting. The contents of figures and all references, patents and published patent applications cited throughout this application are expressly incorporated herein by reference.

### EXAMPLES

### Example 1: Current Pluripotent Culture Media Passively Maintains Pluripotency

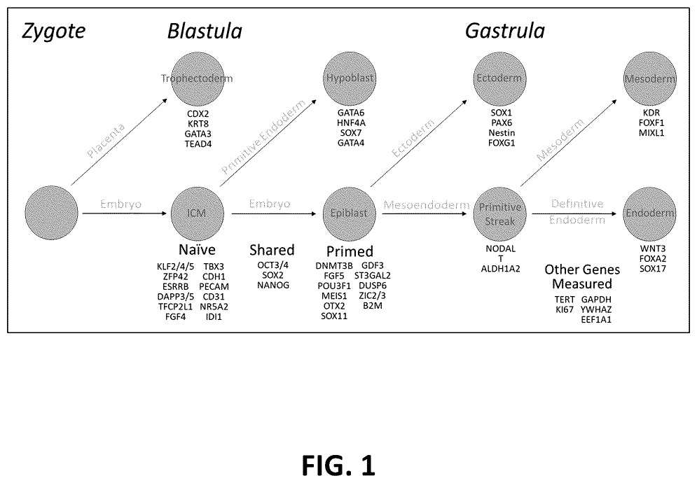

To monitor the differentiation state of a pluripotent culture and track the primed state, naïve state and any early lineage commitment biases that may arise during the culture process, a network consisting of 54 genes was chosen (FIG. 1). Other genes monitored throughout this series of experiments include TERT and KI67. Maximal TERT expression is needed to sustain the pluripotent state in both naïve and primed cells while KI67, a proliferation marker, is expected to increase in the naïve state. Housekeeping genes measured within this QS chip design are used to normalize the individual experimental runs. This network of genes was then monitored using an HD-DoE methodology (Bukys et al. (2020) Iscience 23:101346) as further described in Example 2 to enable in silico modelling of the pluripotent state.

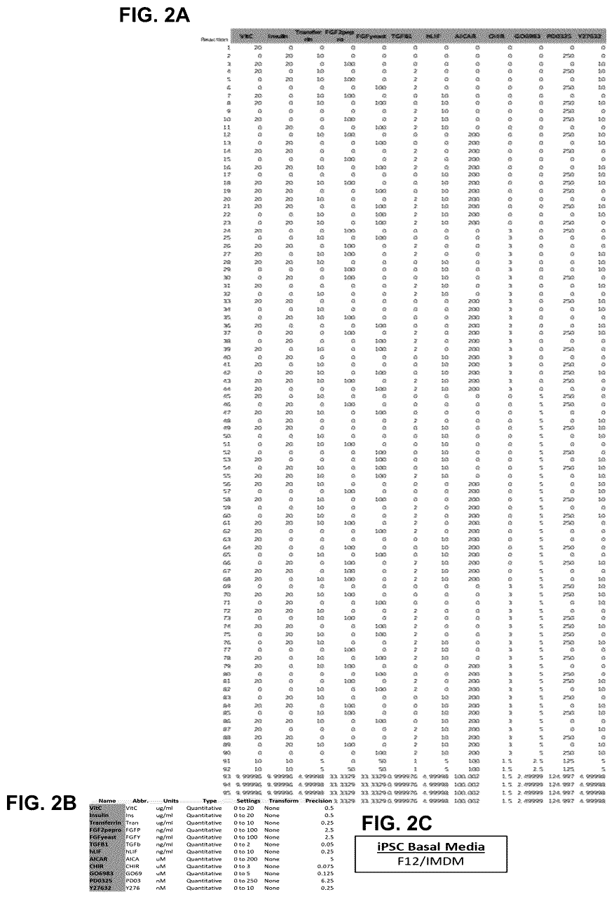

An initial HD-DoE modelling experiment was designed to determine if any of the components within the Essential 8 (E8) media were critical for pluripotent maintenance. The results of this experiment are depicted in FIG. 2. E8 media components include HEPES, bicarbonate, selenium, ascorbic acid, transferrin, insulin, FGF2 and TGF-β. HEPES, bicarbonate and selenium were not considered in this HD-DoE design, because they are additives that are not specific for the maintenance of the pluripotent state but are involved in enabling the overall growth of any cells in culture. In addition, these components are present in most generic media formulations, whereas the initial HD-DoE experiment herein utilized a 1:1 F12/IMDM hybrid media already containing HEPES, bicarbonate and selenium. In addition to the E8 components, human leukemia inhibitory factor (hLIF), AICAR (AMPK pathway agonist), CHIR 99021 (Wnt agonist/GSK-30 antagonist), Go6983 (PKC pathway antagonist), PD0325901 (MEK pathway antagonist), and Y27632 (ROCK inhibitor) were evaluated within this design (FIG. 2). These components were selected based on previous experiments indicating the potential contribution of these components to maintenance of the pluripotent state.

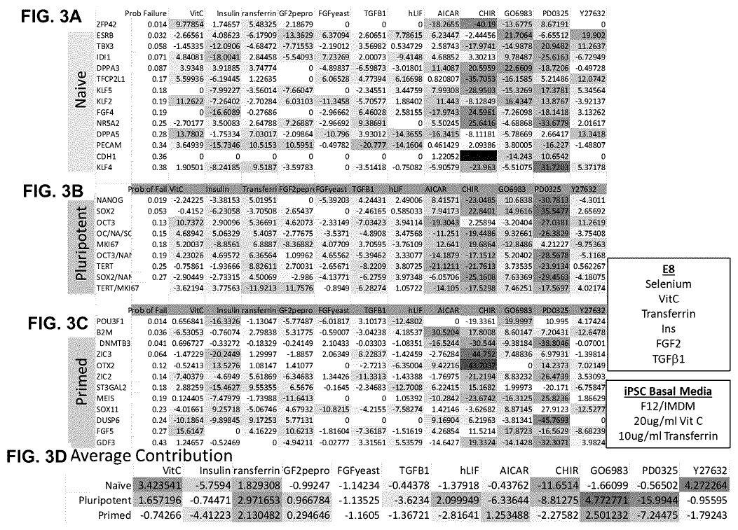

Examination of the overall contributions of the foregoing effectors assayed demonstrated that the only E8 components beneficial in maintaining the pluripotent state were ascorbic acid and transferrin (FIG. 3). The only other components within this design that were determined to potentially aid in pluripotent maintenance were the PKC inhibitor, Go6983 and the ROCK inhibitor, Y27632. Whereas the overall pluripotent state was driven by Go6983, the naïve state was favored by Y27632. All other components within this design presented either conflicting contributions to the pluripotent state or were demonstrated to be clear lineage drivers.

### Example 2: Defining the Critical Signaling Pathways for Pluripotent Maintenance

A data-driven, High-Dimensional Design of Experiments (HD-DoE)-based perturbation of a pluripotent culture was used to assay several cellular signaling pathways known to function within the pluripotent state. The HD-DOE method was applied with the intent to find conditions for directly inducing the naïve state from the pluripotent stem cell state. This example utilizes a method previously described by Bukys et al. (2020) Iscience 23:101346, which employs computerized design geometries to simultaneously test multiple process inputs and provide for mathematical modeling of a deep effector/response space. The method allows for finding combinatorial signaling inputs that control a complex differentiation process and allows testing of multiple plausible critical process parameters impacting output responses, such as gene expression. Because gene expression provides hallmark features of the phenotype of, for example, a human cell, the method can be applied for identifying and understanding the signaling pathways for controlling cell fate.

To develop a cell culture recipe for growing and maintaining PSCs in cell culture and for differentiation of stem cells to naïve state progenitors, the impact of agonists and antagonists of multiple signaling pathways (herein called effectors) on the expression of pre-selected genes was tested and modeled. The impact of each effector on gene expression level is defined by a parameter called factor contribution that is calculated for each effector during the modeling. These effectors are small molecules or proteins that are commonly used promoting differentiation of stem cells to specific fates. Selection of the effectors was based on current literature on differentiation of stem cells to naïve state progenitors.

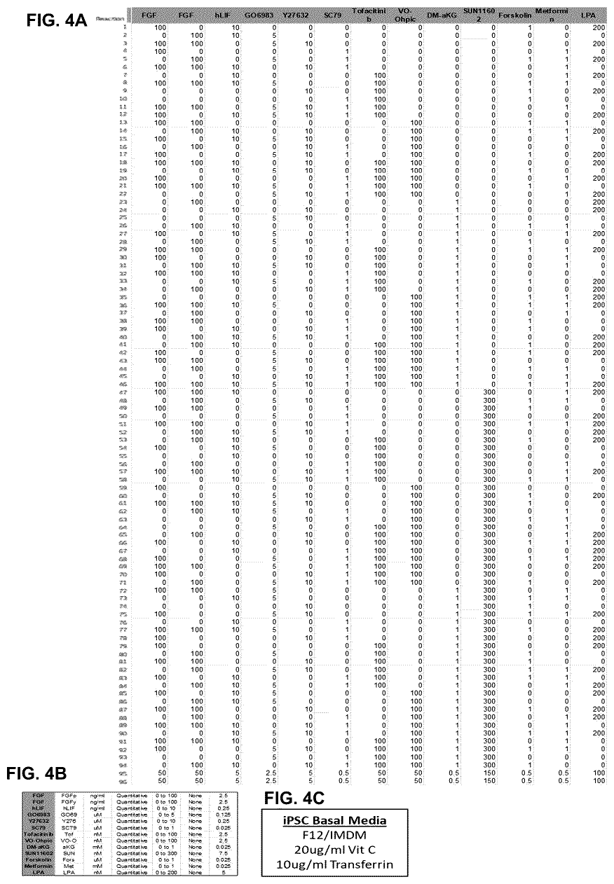

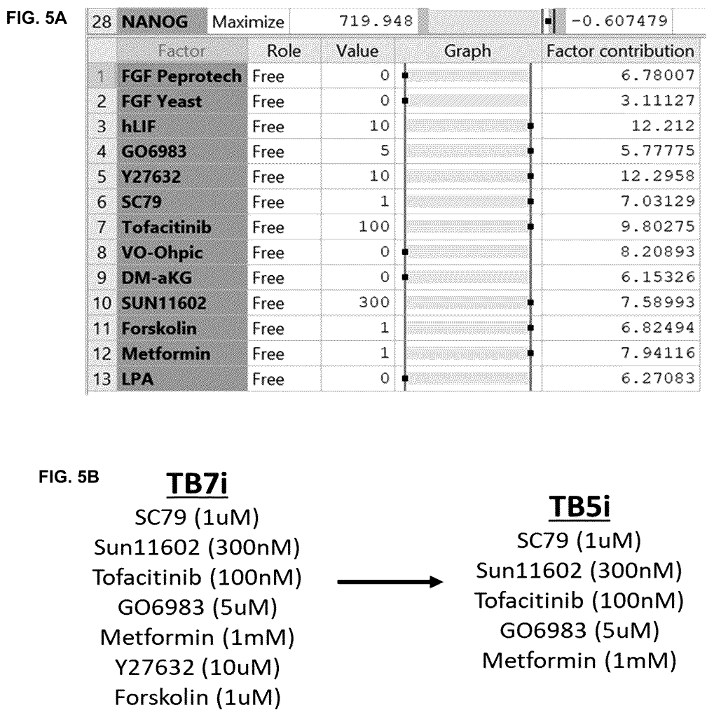

Both the PKC inhibitor GO6983 and the ROCK inhibitor Y27632 were included in the design of the HD-DoE (FIG. 4A-4B). Ascorbic acid and transferrin were added to the basal media for this perturbation matrix (FIG. 4C) and all follow up validation experiments. Focusing on maximizing NANOG as a surrogate for the pluripotent state as well as a key driver of the naïve state, it was determined that a synergistic pathway drive for pluripotency could be achieved through the combined effects of activating the Akt, FGF, AMPK and cAMP pathways, while antagonizing the Jak Stat, ROCK and PKC pathways (FIG. 5A).

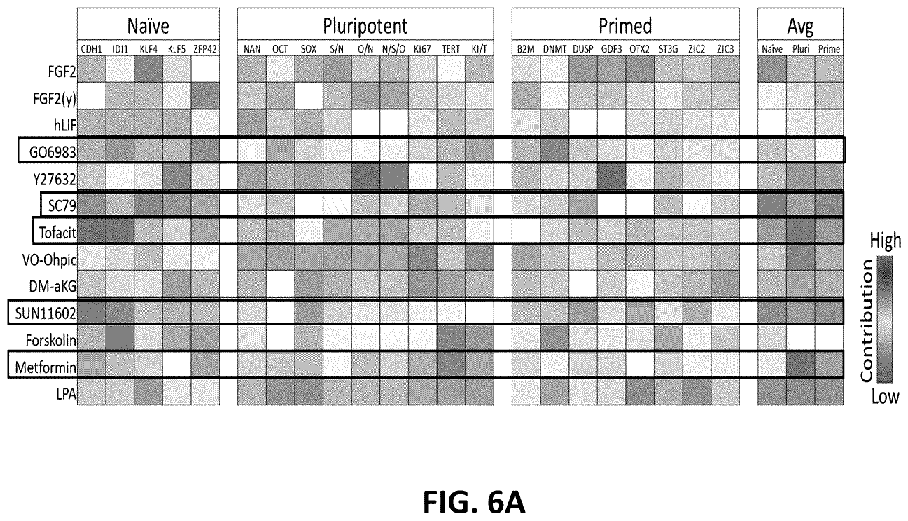

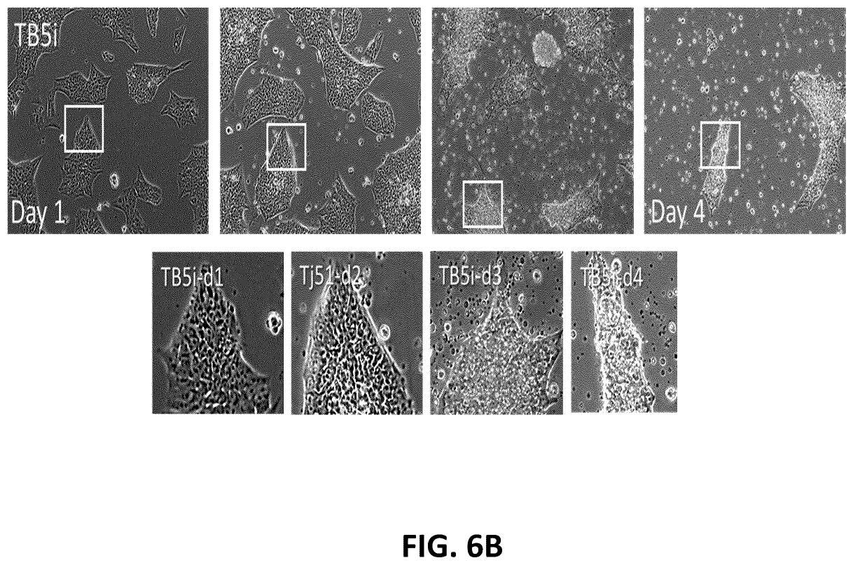

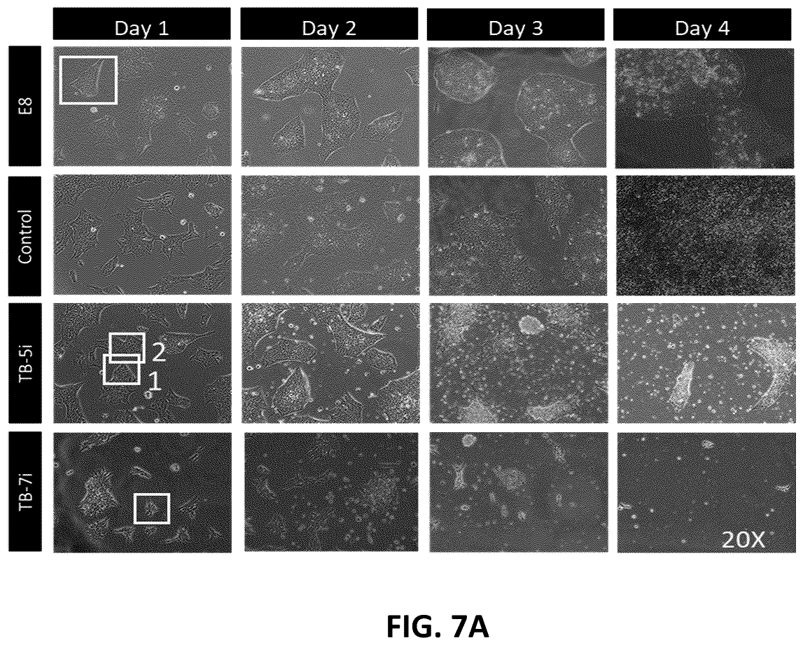

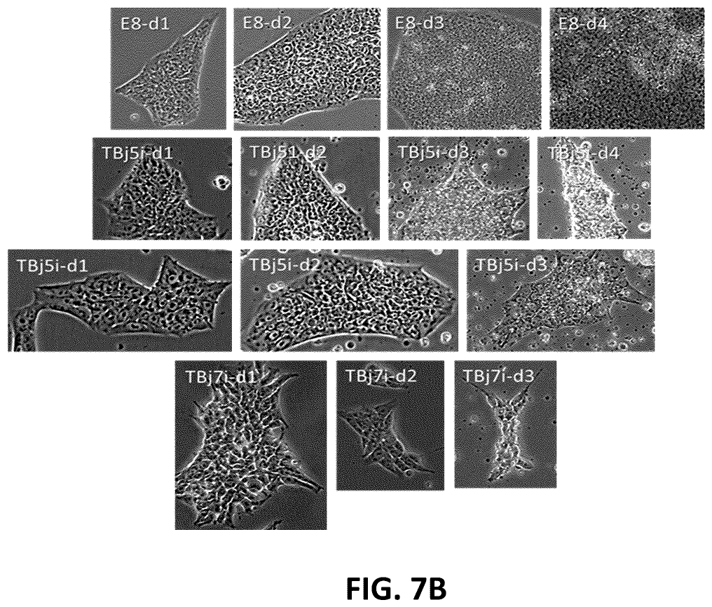

Further analysis through the sequential optimization of all genes measured representative of either the naïve state, primed states or general pluripotent markers suggested that neither the cAMP activator forskolin, nor the ROCK pathway inhibitor were critical to the process (FIG. 6A). Initial validation of the TB5i media demonstrated that colonies quickly began to tightly pack together with a mound phenotype, a well-known characteristic of the naïve state (FIG. 6B). Direct comparison between the TB5i and the TB7i confirmed that neither forskolin, nor Y27632 were beneficial to the overall growth of pluripotent cells (FIG. 7A-7B).

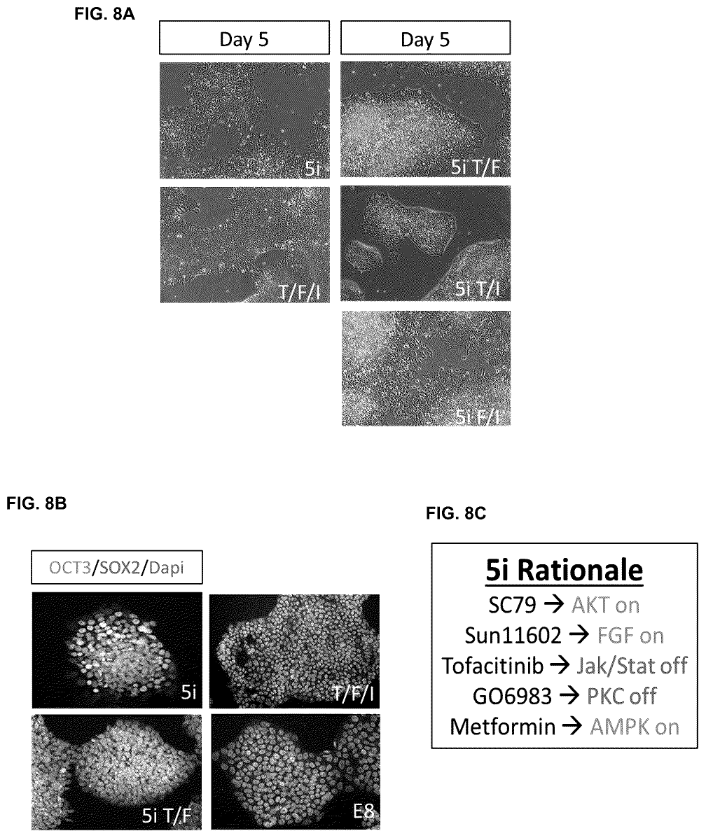

The protein additives FGF2, TGF-β1 and insulin were next assayed as additives in the TB5i formulation (FIG. 8A). It was determined that insulin had little, if any beneficial effect on the culture, thereby confirming the preliminary HD-DoE analysis depicted in FIG. 3D. FGF2 increased the growth rate of the culture, while TGF-β1 sustained normal morphology of the pluripotent colony edges, thereby confirming the passive nature of the Essential 8 formulation. Immuno-histochemical validation for OCT3/4 and SOX2 expression confirmed pluripotent maintenance within the TB5i media. Cultures maintained in both TB5i and TB5i supplemented with TGF-β1 and FGF2 showed a denser phenotype than the control cultures (FIG. 8B).

### Example 3: TB5i Mediated Bioreactor-Based Pluripotent Aggregate Growth

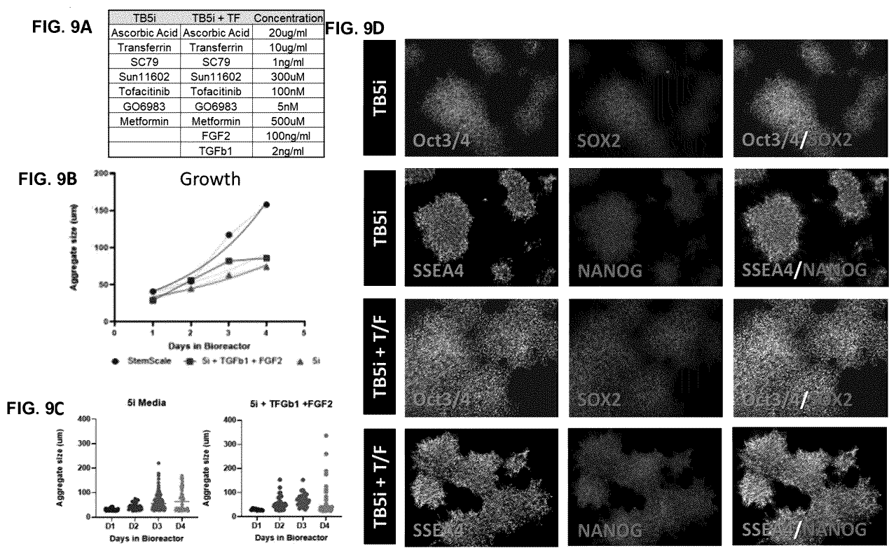

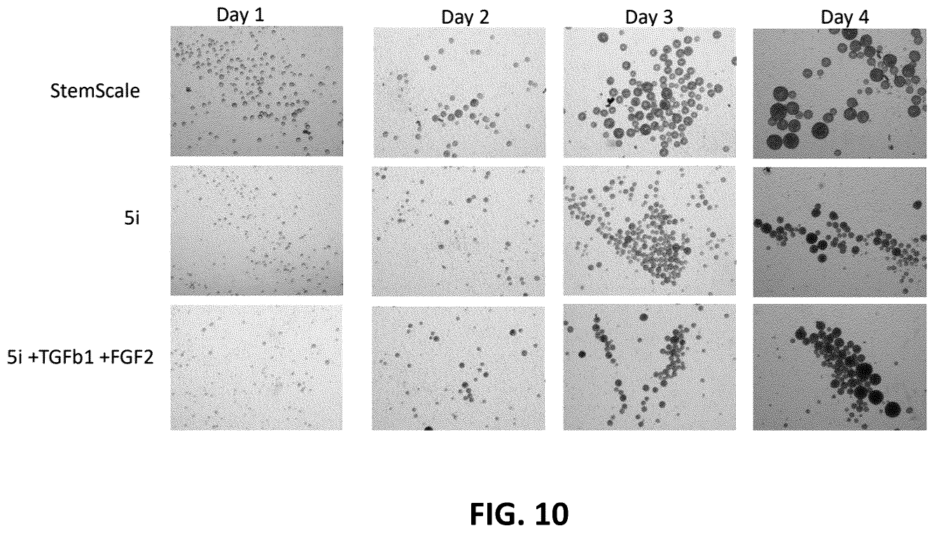

To determine if the TB5i formulation was capable of sustaining pluripotency in a suspension culture, a PBS VW bioreactor system was used. Three experimental conditions were performed. A first bioreactor was used as a control where cells were grown in STEMSCALE™ a proprietary suspension media commercially available (Gibco) for growth of pluripotent cultures in suspension. Second and third bioreactors cells were grown in TB5i or TB5i media supplemented with FGF2 and TGF-β1, respectively (FIG. 9A). Consistent with previous observations (FIG. 8), both cultures grown in TB5i media exhibited growth throughout the bioreactor runs (FIG. 9B) with an increased proliferation for the TB5i supplemented with FGF2 and TGF-β1. Aggregate growth peaked at day 3 (FIGS. 9C & 10A). Validation of the pluripotent state was achieved by plating aggregates for IHC analysis. Pluripotent markers OCT3/4, SOX2, NANOG and SSEA4 were expressed throughout the cultures grown in both conditions (FIG. 9D).

### EQUIVALENTS

Those skilled in the art will recognize or be able to ascertain using no more than routine experimentation, many equivalents of the specific embodiments of the invention described herein. Such equivalents are intended to be encompassed by the following claims.

1. A method for maintenance and expansion of Oct3/4+ SOX2+ NANOG+ pluripotent stem cells (PSCs) in cell culture comprising:
culturing pluripotent stem cells (PSCs) in a culture media comprising an Akt pathway agonist, an FGFR pathway agonist, a JAK/STAT pathway antagonist, a PKC pathway antagonist, and an AMPK pathway agonist such that the culture media maintains the PSCs in a primed or naïve state comprising the markers Oct3/4, SOX2 and NANOG.

2. The method of claim 1, wherein the PSCs are human induced PSCs (hiPSCs).

3. The method of claim 1, wherein the PSCs are human embryonic stem cell (hESCs).

4. The method of claim 1, wherein the Akt pathway agonist is selected from the group consisting of SC79, Demethyl-Coclaurine, LM22B-10, YS-49, YS-49 monohydrate, Demethylasterriquinone B1, Recilisib, N-Oleyol glycine, NSC45586 sodium, Periplocin, CHPG sodium salt, Bilobalide, 6-hydroxyflavone, Musk ketone, SEW2871, 8-Prenylnaringenin, Razuprotafib, and combinations thereof.

5. The method of claim 4, wherein the Akt pathway agonist is SC79, which is present in the culture media at a concentration of 1 ng/ml.

6. The method of claim 1, wherein the FGFR agonist is FGF2 or SUN11602.

7. The method of claim 6, wherein the FGFR agonist is SUN11602, which is present in the culture media at a concentration of 5 μM.

8. The method of claim 1, wherein the JAK/STAT signaling antagonist is selected from the group consisting of Tofacitinib, Ruxolitinib, Baricitinib, Filgotinib, Upadacitinib, Peficitinib, Oclacitinib, Solcitinib, Decernotinib, Delgocitinib, Deucravicitinib, Abrocitinib, Lestaurtinib, Pacritinib, Fedratinib, Momelotinib, Gandotinib, Cerdulatinib, GS-829845, GSK2586184, AZD1480, R348, VX-509, GLPG0634, JSI-124, TG101348, AC-430, NS-018, CHZ868, SHR0302, INCB039110, BMS-911543, BMS-986165, PF-04965841, PF-04965842, PF-06263276, PF-06651600, and combinations thereof.

9. The method of claim 8, wherein the JAK/STAT antagonist is Tofacitinib, which is present in the culture media at a concentration of 100 nM.

10. The method of claim 1, wherein the PKC pathway antagonist is selected from the group consisting of Go6983, Sotrastaurin, Enzastaurin, Staurosporine, LY31615, Go 6976, GF 109203X, Ro 31-8220 mesylate, and combinations thereof.

11. The method of claim 10, wherein the PKC pathway antagonist is Go6983, which is present in the culture media at a concentration of 5 nM.

12. The method of claim 1, wherein the AMPK agonist is selected from the group consisting of Metformin, AICAR, Kazinol B, Marein, Amarogentin, A 769662, PF 06409577, Metformin hydrochloride, ZLN 024, ZLN 024 hydrochloride, Nilotinib, Phenformin, Nilotinib hydrochloride monohydrate, Adenosine 5′-monophosphate monohydrate, Hispidulin, MK 8722, Euphorbiasteroid, ASP4132, GSK621, EX229 (compound 991), Trans-feluric acid, O-304, MK 3903, BAM 15, ligustroflavone, ETC-1002, BC1618, IMM-H007, IM156, Chikusetsusaponin IVa, Poricoic acid A, 7-Methoxyisoflavone, Urolithin B, Danthron, Demethyleneberberine, AMPK activator 1, AMPK activator 2, AMPK activator 4, Malvidin-3-O-arabinoside chloride, RSVA 405, Etilefrin, COH-SR4, Buformin, Buformin hydrochloride, PT1, Bempedoic acid, 3a-Hydrocymogrol, Ampkinone, and combinations thereof.

13. The method of claim 12, wherein the AMPK pathway agonist is Metformin, which is present in the culture media at a concentration of 500 μM.

14. The method of claim 1, wherein the culture media comprises SC79, SUN11602, Tofacitinib, Go6983, and Metformin.

15. The method of claim 14, wherein the culture media comprises 1 ng/ml SC79, 5 μM SUN11602, 100 nM Tofacitinib, 5 nM Go6983, and 500 μM Metformin.

16. The method of claim 1, wherein the culture media further comprises a TGF-β1 agonist.

17. The method of claim 16, wherein the TGF-β1 agonist is selected from the group consisting of TGF-β1, SRI-011381, Activin A, Nodal, DPS-1, and combinations thereof.

18. The method of claim 17, wherein the TGF-β1 agonist is TGF-β1, which is present in the culture media at a concentration of 2 ng/ml.

19. The method of claim 1, wherein the culture media further comprises a basal media composition selected from the group consisting of DMEM, F12, IMDM, CDM2, and combinations thereof.

20. The method of claim 19, wherein the basal media composition is further supplemented with ascorbic acid and transferrin.

21. The method of claim 20, wherein the basal media composition comprises 1:1 F12/IMDM media supplemented with 20 μg/ml ascorbic acid, 10 μg/ml transferrin and 1% penicillin-streptomycin.

22. The method of claim 20, wherein the basal media composition comprises selenium, ascorbic acid, transferrin, FGF2 and TGF-β1.

23. The method of claim 1, wherein the PSCs are human CD7+ CD75+ CD77+ CD130+ F11R+ naïve pluripotent cells.

24. The method of claim 1, wherein the PSCs express KLF2/4/5, ZFP42, ESRRB, DAPP3/5, TFCP2L1, FGF4, TBX3, CDH1, PECAM, CD31, NR5A2, and IDID1.

25. The method of claim 1, wherein the PSCs are grown in an adherent culture format.

26. The method of claim 25, wherein the adherent culture format comprises growing the PSCs on a tissue culture plate coated with gelatin, vitronectin, MATRIGEL® or GELTREX®.

27. The method of claim 1, wherein the PSCs are grown in a suspension culture as a cell aggregate.

28. The method of claim 27, wherein the PSCs are grown in a bioreactor.

29. A method for generating and maintaining human CD7+ CD75+ CD77+ CD130+ F11R+ naïve pluripotent cells in cell culture comprising:
culturing human pluripotent stem cells (PSCs) in a culture media comprising an Akt pathway agonist, an FGFR pathway agonist, a JAK/STAT pathway antagonist, a PKC pathway antagonist, an AMPK pathway agonist, a ROCK inhibitor, and a TGF-β1R agonist, such that the culture media generates and maintains a human CD7+ CD75+ CD77+ CD130+ F11R+ naïve pluripotent cell in culture.

30. A culture media for maintenance and expansion of Oct3/4+ SOX2+ NANOG+ pluripotent stem cells (PSCs) in cell culture, comprising: an Akt pathway agonist, an FGFR pathway agonist, a JAK/STAT pathway antagonist, a PKC pathway antagonist, and an AMPK pathway agonist.

31. The culture media of claim 30, wherein the Akt pathway agonist is selected from the group consisting of SC79, Demethyl-Coclaurine, LM22B-10, YS-49, YS-49 monohydrate, Demethylasterriquinone B1, Recilisib, N-Oleyol glycine, NSC45586 sodium, Periplocin, CHPG sodium salt, Bilobalide, 6-hydroxyflavone, Musk ketone, SEW2871, 8-Prenylnaringenin, Razuprotafib, and combinations thereof.

32. The culture media of claim 31, wherein the Akt pathway agonist is SC79.

33. The culture media of claim 30, wherein the FGFR agonist is FGF2 or SUN11602.

34. The culture media of claim 33, wherein the FGFR agonist is SUN11602.

35. The culture media of claim 30, wherein the JAK/STAT signaling antagonist is selected from the group consisting of Tofacitinib, Ruxolitinib, Baricitinib, Filgotinib, Upadacitinib, Peficitinib, Oclacitinib, Solcitinib, Decernotinib, Delgocitinib, Deucravicitinib, Abrocitinib, Lestaurtinib, Pacritinib, Fedratinib, Momelotinib, Gandotinib, Cerdulatinib, GS-829845, GSK2586184, AZD1480, R348, VX-509, GLPG0634, JSI-124, TG101348, AC-430, NS-018, CHZ868, SHR0302, INCB039110, BMS-911543, BMS-986165, PF-04965841, PF-04965842, PF-06263276, PF-06651600, and combinations thereof.

36. The culture media of claim 35, wherein the JAK/STAT antagonist is Tofacitinib.

37. The culture media of claim 30, wherein the PKC pathway antagonist is selected from the group consisting of Go6983, Sotrastaurin, Enzastaurin, Staurosporine, LY31615, Go 6976, GF 109203X, Ro 31-8220 mesylate, and combinations thereof.

38. The culture media of claim 37, wherein the PKC pathway antagonist is Go6983.

39. The culture media of claim 30, wherein the AMPK agonist is selected from the group consisting of Metformin, AICAR, Kazinol B, Marein, Amarogentin, A 769662, PF 06409577, Metformin hydrochloride, ZLN 024, ZLN 024 hydrochloride, Nilotinib, Phenformin, Nilotinib hydrochloride monohydrate, Adenosine 5′-monophosphate monohydrate, Hispidulin, MK 8722, Euphorbiasteroid, ASP4132, GSK621, EX229 (compound 991), Trans-feluric acid, O-304, MK 3903, BAM 15, Ligustroflavone, ETC-1002, BC1618, IMM-H007, IM156, Chikusetsusaponin IVa, Poricoic acid A, 7-Methoxyisoflavone, Urolithin B, Danthron, Demethyleneberberine, AMPK activator 1, AMPK activator 2, AMPK activator 4, Malvidin-3-O-arabinoside chloride, RSVA 405, Etilefrin, COH-SR4, Buformin, Buformin hydrochloride, PT1, Bempedoic acid, 3a-Hydrocymogrol, Ampkinone, and combinations thereof.

40. The culture media of claim 39, wherein the AMPK pathway agonist is Metformin or AICAR.

41. The culture media of claim 30, which comprises SC79, SUN11602, Tofacitinib, Go6983, and Metformin.

42. The culture media of claim 30, which further comprises a TGF-β1 agonist.

43. The culture media of claim 42, wherein the TGF-β1 agonist is selected from the group consisting of TGF-β1, SRI-011381, alantolactone, Activin A, Nodal, DPS-1, and combinations thereof.

44. The culture media of claim 43, wherein the TGF-β1 agonist is TGF-β1 or SRI-011381.

45. The culture media of claim 30, which further comprises a basal media composition selected from the group consisting of DMEM, F12, IMDM, CDM2, and combinations thereof.

46. The culture media of claim 45, wherein the basal media composition is further supplemented with ascorbic acid and transferrin.

47. The culture media of claim 46, wherein the basal media composition comprises F12 or IMDM media supplemented with ascorbic acid, transferrin, and penicillin-streptomycin.

48. The culture media of claim 46, wherein the basal media composition comprises selenium, ascorbic acid, transferrin, FGF2, and TGF-β1.
